(19) United States
(12) Patent Application Publication
(10) Pub. No.: US 2022/0170087 A1
(43) Pub. Date: Jun. 2, 2022

(54) Method of DNA Base-Calling from a Nanochannel DNA Sequencer

(71) Applicants: Steve TUNG, Fayetteville, AR (US); Bo MA, Fayetteville, AR (US); Board of Trustees of the University of Arkansas, Little Rock, AR (US)

(72) Inventors: Steve Tung, Fayetteville, AR (US); Bo Ma, Fayetteville, AR (US)

(21) Appl. No.: 17/441,094

(22) PCT Filed: Mar. 18, 2020
(86) PCT No.: PCT/US2020/023283

§ 371 Date: Sep. 20, 2021

Related U.S. Application Data
(60) Provisional application No. 62/819,783, filed on Mar. 18, 2019.

(51) Int. Cl. C12Q 1/6869; G01N 27/04; G01N 33/487

(52) U.S. Cl. CPC C12Q 1/6869; G01N 33/48721; G01N 27/04

(57) ABSTRACT

A method of DNA base-calling from a nanochannel DNA sequencer. The method includes building a reference map and preparing an unknown sequence of DNA prior to the final step of data matching. The reference map includes a series of reference characters, such as numbers, that describe the change in tunneling current of a DNA strand with a known sequence. A DNA strand of unknown sequence is prepared so that the change in electrical measurement can also be described numerically. The section of match between the DNA strand of unknown sequence and the reference map is used to determine the sequence of the DNA strand.

### CROSS-REFERENCE TO RELATED APPLICATIONS

This application claims the benefit of U.S. Provisional Application No. 62/819,783, entitled “Method for DNA Base-Calling from a Nanochannel DNA Sequencer” and filed on Mar. 18, 2019. The complete disclosure of said provisional application is hereby incorporated by reference.

### STATEMENT REGARDING FEDERALLY SPONSORED RESEARCH OR DEVELOPMENT

This invention was made with government support from grant no. 1128660 awarded by the National Science Foundation and grant no. 1R21HG010055-01 awarded by the National Institute of Health. The government has certain rights in the invention.

### BACKGROUND ART

Advanced DNA sequencing technologies have given great optimism to the future of public health. These technologies provide vital information to support research throughout the field of disease diagnoses, prevention, and treatment. To sequence a complete human genome, which contains about 3 billion base pairs (bp), current sequencing technologies such as the Next Generation or Third Generation DNA sequencing require the DNA sample to be chopped into short segments. Through the approach of massively parallel processing, the human genome sequencing can be accomplished in weeks, however, the high cost of the facility and long lead time of sequencing persists due to the short reading length of each segment.

The world's first nanopore DNA sequencer, MinION from ONT (Oxford Nanopore Technologies) is based upon the technology of blockage current. Theoretically, when DNA is translocating through the nanopore, ionic current through the nanopore is blocked by the presence of DNA. The amplitude of blockage current depends on the interaction between the DNA bases and nanopore. However, existing nanopore configurations are relatively thick (a few nanometers) and measure the blockage current induced by multiple DNA bases instantaneously. Raw data performance assessments show that initially the ONT MinION achieved only a 60-70% sequencing accuracy because of the thickness of the nanopores.

A method of fabricating nanochannel systems for DNA sequencing and nanoparticle characterization is disclosed in U.S. Pat. No. 9,718,668 (Steve Tung et. al). While the patented method made important strides in the field of DNA sequencing, the patented method fails to address one critical challenge for the application of DNA sequencing: existing technologies (like the MinION ONT, for example) are not suitable for analyzing the tunneling current measured by nanoelectrodes with a width wider than a single DNA base (about 0.3 nm). Because of this issue, existing methods do not allow for the direct reading of the DNA sequence from the tunneling measurement based on its amplitude.

Furthermore, while ONT software has been developed further to increase sequencing accuracy, such software cannot be used to analyze data generated using devices such as the one described in U.S. Pat. No. 9,718,668 because (a) fundamentally the measurement mechanism is different; (b) the ONT software is based on the algorithms of deep learning, which cannot be adopted to current uses for the sequencing data training step; and (c) using nanoelectrode to measure transverse current involves DNA orientation considerations that were not considered in the ONT algorithms. While the basic concept of data processing is common (i.e. to reveal the DNA sequence information based on their context), a novel DNA base-calling method for tunneling current analysis is necessary to address these challenges.

### DISCLOSURE OF THE INVENTION

The present invention is directed to a method for DNA base-calling from a nanochannel DNA sequencer. Base-calling is a process that converts raw signals into readable DNA sequences. The process consists of two major tasks (building a reference map and preparing experimental data) prior to the final step of data matching. In the present invention, the reference map refers to a series of numbers built based on a standard DNA sequence to describe the change of its corresponding tunneling current. Experimental data is prepared so that the change of electrical measurement can be described numerically. A section of match between the prepared experimental data and the reference map is used for DNA base-calling. The present invention utilizes seven sequential steps to execute these two major tasks, with mathematical models developed to accomplish the goal of each of the sequential steps. The novel DNA translocation protocol of the present invention utilizes AFM (atomic force microscope) based nanomanipulation to select and pick a single DNA molecule from a substrate surface. By moving the AFM tip in an aquaria environment, the DNA is stretched to linear during the process of DNA tunneling current measurement. This process is essential for allowing the DNA sequence to be output as the final results.

These and other objects, features, and advantages of the present invention will become better understood from a consideration of the following detailed description of the preferred embodiments and appended claims in conjunction with the drawings as described following:

### BRIEF DESCRIPTION OF THE DRAWINGS

FIG. 1 is a diagram showing an overview of the DNA base-calling process for DNA sequencing of the present invention.

FIG. 2A shows a representation of DNA translocating through single-chain nanoelectrodes and FIG. 2B shows the corresponding tunneling current.

FIG. 3A shows DNA translocating through triple-chain nanoelectrodes and FIG. 3B shows the corresponding tunneling current.

FIG. 4A shows the analytical method of matching DNA translocating with a tunneling current with the assistant of ‘moving window’ and FIG. 4B shows the trends associated with the tunneling current thereof.

FIG. 5A shows an example of a DNA sequence in a forward translocation direction and FIG. 5B shows the example DNA sequence in a backward translocation direction.

FIG. 6A shows the example DNA sequence of FIG. 5 converted into a double strand DNA in a forward translocation direction and FIG. 6B shows the example DNA sequence converted into a double strand DNA in a backward translocation direction.

FIG. 7 shows the effect of a DNA base pair rotating its orientation by 36 degrees per base pair and mapping the rotation as a sin wave function.

FIG. 8 shows an example of converting a section of dsDNA sequence in an array to three possible conductance arrays.

FIGS. 9A-9B are schematics showing two extreme cases and their equivalent circuits when a DNA base pair of A-T is translocating through the gap between nanoelectrodes. FIG. 9A shows the 90° orientation and its equivalent parallel circuit and FIG. 9B shows the 0° orientation and its equivalent series circuit.

FIG. 10 is a table showing the conductance equation selections for corresponding base pair orientations.

FIG. 11 shows an example of dsDNA base pairs along the double helix structure changing their orientation to nanoelectrodes during the process of DNA translocation.

FIG. 12 is a table showing the known relative conductance of each type of nucleotide.

FIG. 13 is a schematic showing the concept of converting the conductance induced by each DNA base pair to a transverse conductance measurement.

FIG. 14 shows an example of a reference map of DNA in forward translocation direction.

FIG. 15A shows raw data and FIG. 15B shows processed data for the transverse current measurement when DNA translocates through a nanoelectrode at 0.1 μm/s.

FIG. 16A shows an example tunneling current signal graph and FIG. 16B shows the DNA section believed to be stretched zoomed in and coded numerically.

FIG. 17 is a graph showing the matching result of experimental DNA sequencing data to the reference map.

FIG. 18 is a graph showing the DNA sequencing raw data and the coding result at the beginning of the signal.

FIG. 19 shows the format of organizing sequence data for the VISTA submission.

FIG. 20 shows the VISTA mapping result. The highlighted sequence is used to match the experimental data.

FIG. 21 is a data plot showing the experimental data (current as a function of time) divided into seven sections (A-G).

FIG. 22 is a graph showing the reference data matched to experimental data in four separated sections and assembly using the VISTA tool kit.

### BEST MODE FOR CARRYING OUT THE INVENTION

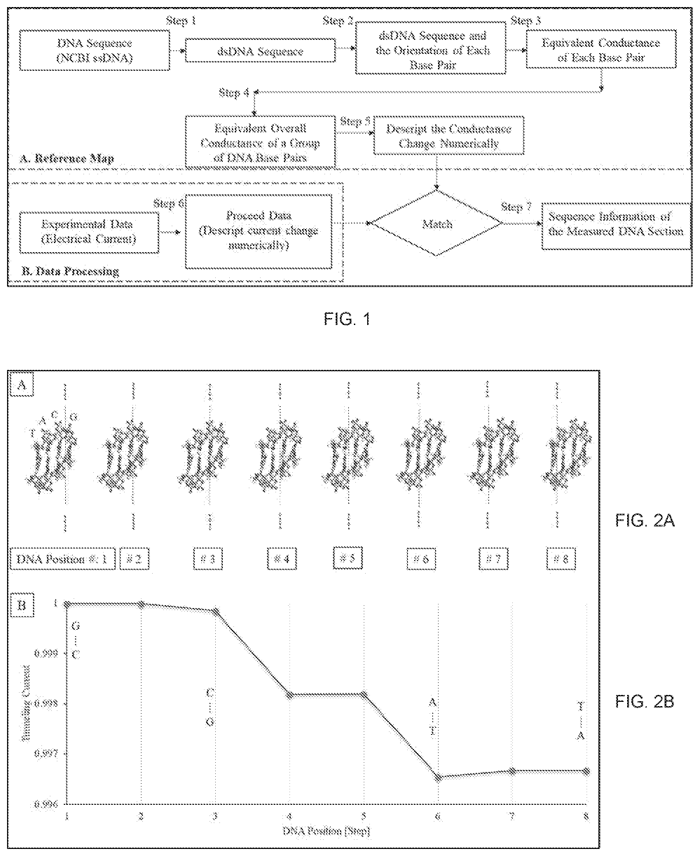

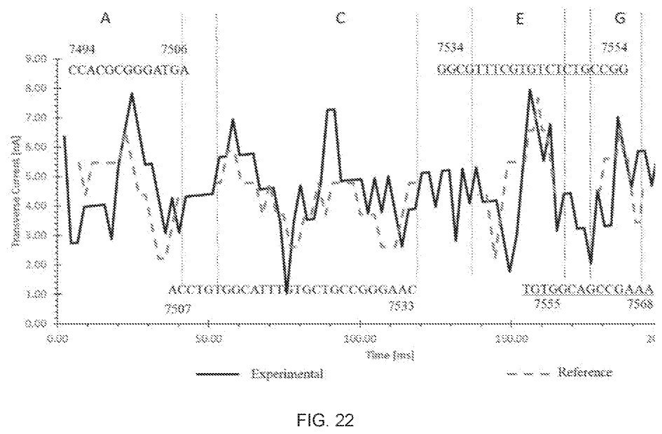

With reference to FIGS. 1-22, the present invention may be described. The present invention is directed to a method for DNA base-calling from a nanochannel DNA sequencer. As noted above, base-calling requires two major tasks: reference mapping and data processing. In the present invention, these major tasks are executed in a series of seven sequential steps (as shown in FIG. 1). Steps 1-5 are useful for executing the reference mapping task and step 6 is useful for the data processing task. Step 7 calls upon the outputs of the reference mapping task and data processing task in a matching step that allows for the sequence information of the measured DNA to be the final output.

Experimental Background and Method Development: Before describing the method of DNA base-calling embodied by the present invention, it should be noted that the improved DNA base-calling process of the present invention is based off experimental analysis that used quantum simulation to investigate the effect various DNA base pairs have on tunneling current measurement. Such experimental process used for deriving the method of the present invention is described here for background purposes. For best results, the quantum simulation was first performed using a single chain-like Pt nanoelectrode. For simulation purposes, DNA having a sequence of GCAT (top strand reading from right to left) was used as a model. This DNA model is shown in FIG. 2A. In this model, dsDNA is translocating from the left side of the nanoelectrode to the right side in total of eight steps with a 1 Å moving interval, as shown in FIG. 2A. The resulting tunnel current is summarized in FIG. 2B. As shown, the experimental results show that there is no significant difference in terms of the tunneling current measurement for an A-T pair and T-A pair. Likewise, there is no significant difference for a G-C pair and a C-G pair. Based on this conclusion, the DNA sequence with four base pairs (A-T pair, T-A pair, C-G pair, and G-C pair) could theoretically be equivalent to a sequence with two base pairs (A-T pair, and C-G pair). Results also show that G-C and C-G pairs are generally more conductive than A-T and T-A pairs.

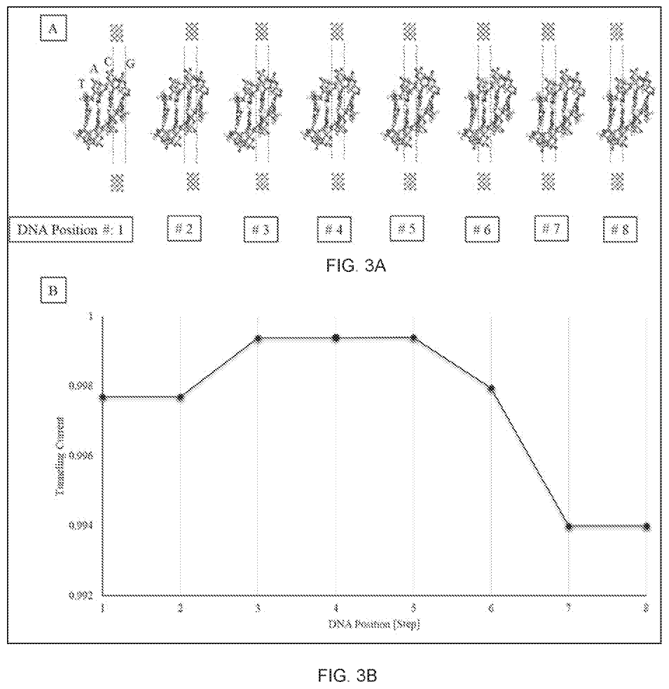

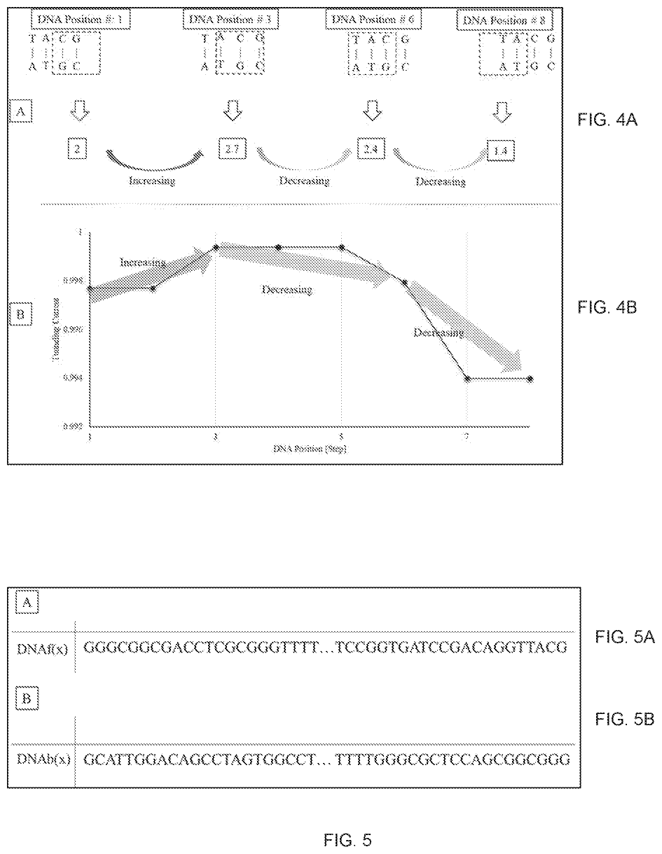

A further simulation with the goal of analyzing tunneling current without single base resolution was performed. The same DNA model as the first simulation was used and the width of the nanoelectrodes were increased from single chain to triple chain as shown in FIG. 3A. Simulation results, shown in FIG. 3B, demonstrate that the relationship between the normalized tunneling current and the DNA base pairs are not clear. Thus, it was determined that no DNA sequencing data can be directly ‘read’ based on the tunneling current amplitude. Thus, it was determined that the non-single base sequencing resolution requires an additional step of data processing for DNA base calling. A concept of a “sliding window” was developed to describe the moving of DNA through the detecting range of nanoelectrodes. To do so, the DNA model was assigned with a box that had a length equivalent to a 3 bp long DNA segment. Based on model configuration, the 3 bp length was determined based on the detection range of the nanoelectrodes (4.2 Å). To trace the change, DNA base pairs composition inside of the sliding window during the DNA translocation were changed and the process of DNA translocation was assembled as shown in FIG. 4A. From the DNA Position #1 to DNA Position #3, the A-T pair came into the detection range and increased the tunneling current. From the DNA Position #3 to Position #6, the leading pair, the G-C pair, was swapped out by the T-A pair. Because T-A pair was shown to be less conductive than the G-C pair, the tunneling current was expected to decrease slightly. Then, from DNA position #6 to position #8, only two DNA base pairs were left in the detection range and a rapid decline in tunneling current was expected, as shown in FIG. 4B. The moving DNA strand therefore can be correlated with the tunneling current change rather than the amplitude of the tunneling current. Based on the simulation results, with the assistance of a ‘moving window’, the change of tunneling current showed a clear correlation to the sequence of tested DNA. Since the ultra slow DNA translocation speed could make the tunneling current sequence-dependent, the base call method of the present invention (described below) was developed to analyze the experimental data for DNA sequencing.

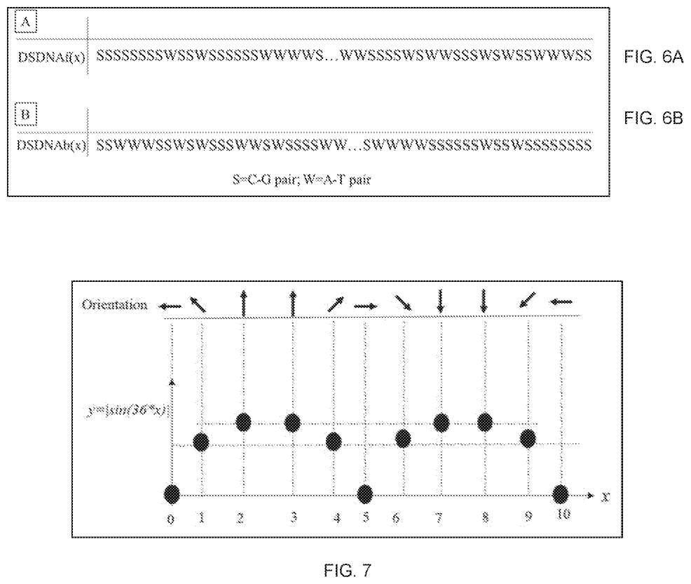

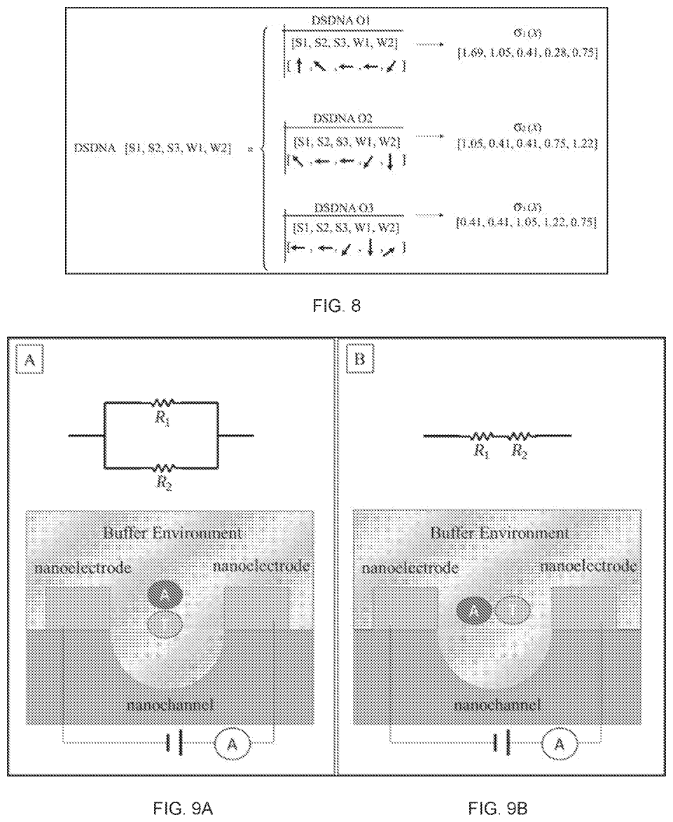

DNA Base-Calling Process: The process begins with a single-stranded DNA (ssDNA) sequence. At step 1, the ssDNA is converted into a double-stranded DNA (dsDNA) sequence based on the ssDNA sequence. A dedicated notation is used to describe the base pair information along the DNA strand. For example, step 1 may use the basic DNA base pairing principle (A to T and C to G) to complement the double stranded DNA (dsDNA). It may be seen then, that an ssDNA sequence having the sequence shown in the forward translocation direction FIG. 5A, for example, will, in the backward translocation direction, have the sequence shown in FIG. 5B. Based off basic DNA base pair principles, the ssDNA sequence above may be converted into a dsDNA sequence having the sequence shown in FIG. 6A in the forward translocation direction and the sequence shown in FIG. 6B in the backward translocation direction. For exemplary purposes, the notation CS' may represent a ‘C-G’ pair and the notation ‘W’ represents an ‘A-T’ pair. In one embodiment, programming (such as MATLAB) may be used to convert the reference ssDNA sequence into the appropriate dsDNA sequence using arrays and matrices to appropriately execute base pairing.

When a translocating DNA strand hits a nanoelectrode gap, each DNA base pair will interact with the nanoelectrodes in a particular orientation. When a dsDNA is translocating through a pair of patterned nanoelectrodes, polarization of the DNA base pair and the direction of tunneling current will vary depending on the base pair's particular orientation. Theoretically, during DNA translocation, the orientation of each base pair is determined by its position along the DNA double helix structure. dsDNA has a helix structure and each base pair twists at an angle of 36 degrees. Thus, a complete 360 degree turn is achieved every 10 DNA base pairs. The effect of this orientation change can be represented by a sine wave as shown in FIG. 7. The horizontal axis is the position of DNA base pair along the double helix structure, and the vertical axis is the value of the sine wave function.

For experimental purposes in developing the invention, and based on the fact that every 10 base pairs completes a 360 degree turn, experimental base pairs were described by one of three orientations (0°, 36°, and 72°). To simplify the analysis even further, orientations of 0°, 36°, and 72° were approximated to 0°, 45°, and 90° to accommodate the theory of using equivalent circuits. These approximated orientations are shown in FIG. 7. These three orientations (O₁, O₂, and O₃) are represented by the following equations, where x represents the index:

$${O}_{1} ( x ) = abs ( sin ( 36 * x ) )$$

$${O}_{2} ( x ) = abs [ sin ( 36 * ( x + 1 ) ) ]$$

$${O}_{3} ( x ) = abs [ sin ( 36 * ( x + 2 ) ) ]$$

Using this process, the orientation of each of the DNA base pairs can be successfully described numerically to describe its periodical property. It should be noted, of course, that in actual practice of the invention described herein the orientation of the first DNA base pair determines the orientation of all of the base pairs that follow. It may be noted, then, that the simplistic experimental view of the base-pairs of 0°, 36°, and 72° (approximated to 0°, 45°, and 90°) may no longer apply. Instead, in practice, the orientation of each DNA base pair along the strand is determined using the orientation of the leading pair, and the orientation of the leading pair can fall anywhere in the range of 0° to 360°. If the leading pair is 0°, for example, the second pair will be 36°. If, however, the leading pair is 1.5°, the second pair will be 37.5°. At step 2, a matrix is built and contains the dsDNA sequence in the first row and the orientation of each corresponding base pair in the second row. Using the dsDNA sequence of base pairs and the orientation of each base pair, matrices can be established. For example, for a short piece of ssDNA with a sequence of GCGTA, a dsDNA sequence of SSSWW may be determined based off of basic base pairing principles (as described above). Assuming the orientations of the first base pair is one of 0°, 36°, and 72° (which are approximated to 0°, 45°, and 90°), three rows of a matrix or three individual matrices may be generated (An example is shown in FIG. 8). The different matrices (DSDNA O₁, O₂, and O₃) represent the three possible configurations of DNA and nanoelectrodes. In each matrix, the first line shows the sequence of the dsDNA (in either forward or backward moving direction) and the second line contains the orientation information of each DNA base pair with a same vertical index. For ease of describing the invention, the matrices in FIG. 8 show arrows indicating orientation, but in computational processing, the second row of each matrix contains numbers obtained through the sine wave function as described above. It should be noted, of course, that the particular sequence described herein (GCGTA) is exemplary only and the present invention may be useful for analysis of any sequence. Likewise, while for purposes of describing the invention the same simplistic assumptions for base pair orientation are presented, in practice, the orientation of the first base pair (which may be anywhere from 0 to 360 degrees) is used to determine the true orientation of the remaining base pairs.

At step 3, the DNA base pair information and its orientation are combined and an equivalent conductance for each base pair is generated. To calculate the equivalent conductance for each base pair, equivalent circuits are used to refer to corresponding base pair orientations to the nanoelectrodes, as shown in FIGS. 9A-B. The top and bottom limit of conductance due to the DNA translocation can be quantified by setting the orientation of a DNA base pair to 90° and 0°, respectively. These two orientations are equivalent to a parallel and series circuit as shown in FIGS. 9A and 9B, respectively (using a base pair of A-T as an example).

Based on the conductance equations of the parallel and series circuit, the equivalent conductance of each base pair may be calculated given the base pair's orientation to the nanoelectrodes using the following equations:

$${G}_{p} = \frac{1}{{R}_{1}} + \frac{1}{{R}_{2}} \\ {G}_{s} = \frac{1}{{R}_{1} + {R}_{2}}$$

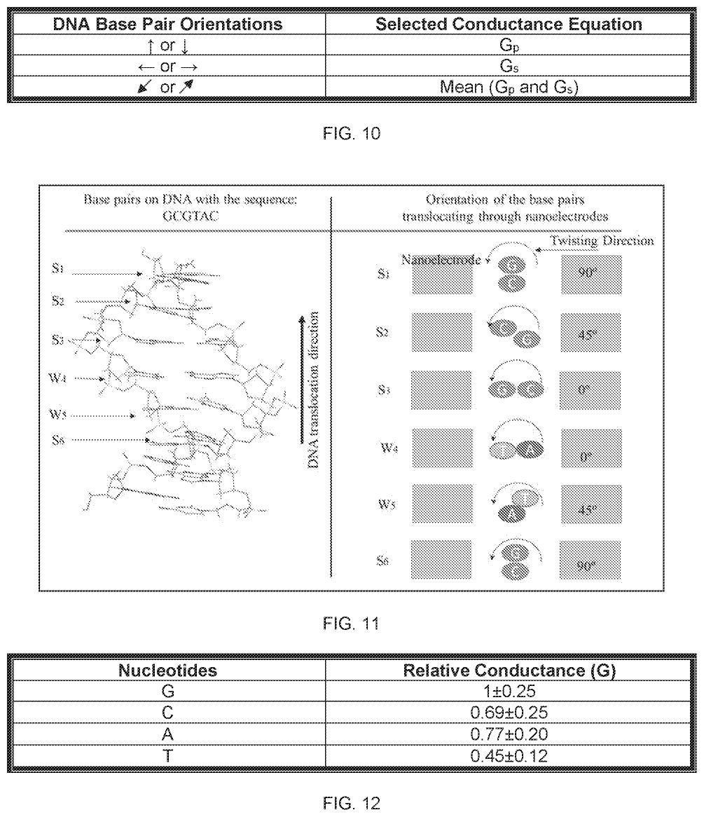

The equations provided above (where Gₚ refers to the conductance of an equivalent parallel circuit and Gₛ refers to the conductance of an equivalent series circuit) are used to calculate the equivalent conductance of each base pair based on the relationships shown in FIG. 9 and represented in FIG. 10. In the above equations, 1/R₁ is the conductance of one of the DNA bases and 1/R₂ is that of the other DNA base in the base pair.

To better understand the relationship between the position of a DNA base pairs to their orientation, consider the following example using a dsDNA with the sequence of GCGTAC, where the first base pair is assumed to be in the 90 degree orientation. As noted above, the notations S and W may be used to refer to particular base pairs (G-C and A-T, respectively). Subscripts may be used to indicate the position of the base pair along the double helix structure. When this DNA section was translocated through the nanoelectrodes, the double helix structure twists the orientation of the DNA base pairs in steps of 36° for each (rounded to 45°) as previously described, and as shown in FIG. 11. The equivalent conductance of S1, S3, W4, and S6 are determined by using the parallel (Gₚ) and series (Gₛ) equations above according to their orientations. In contrast, the equivalent conductance of S₂ and W₅ are determined by first calculating the conductance of that base pair by using both the parallel and series equations, and then taking a mean of those two values. These data processing steps are repeated for each DNA base pair along the sequence of reference DNA for reference map construction. Once the orientation and sequence information is combined and the calculations of conductance are complete the DNA sequence (for example, GCGTA) is converted into three possible conductance arrays: σ₁(x), σ₂(x), and σ₃(x) by using the conductance number of each nucleotides listed in FIG. 12. In practice, conductance arrays will correspond to each possible orientation sequence for each translocation direction.

At step 4, the system conductance is produced using the conductance of each DNA base pair. The system conductance is defined as the conductance that should be theoretically detected by the nanoelectrodes. Due to the width of the nanoelectrode detection range, the system conductance may be calculated by combining multiple DNA base pairs simultaneously. The number of DNA base pairs that should be included in this calculation is determined by the ‘window’ size as described previously. The equivalent system conductance is determined by combining the conductance of each DNA base pair based on the physical properties of the experimental setup to simulate the measured tunneling current. The conductance arrays generated through step 3 consist of the conductance of each individual DNA base pair.

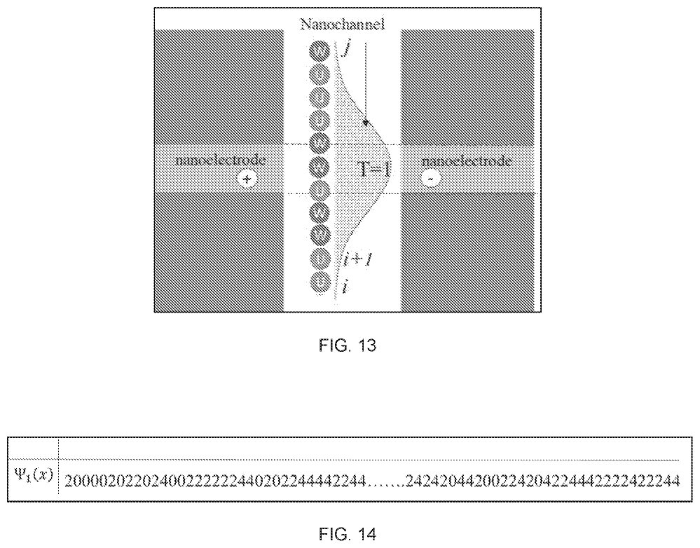

In practice, each instantaneous tunneling measurement is composed by the tunneling effect of multiple DNA base pairs due to the large width of the nanoelectrodes. FIG. 13 demonstrates an additional step required to convert the conductance listed in any array into a format that represents the instantaneous measured conductance due to the measure of conductance being attributed to multiple DNA base pairs. When a section of dsDNA translocates through the gap of nanoelectrodes, as shown in in FIG. 13, DNA base pairs from i to j contribute to the conductance measurement simultaneously. Based on its location and the distance to the center of detection range, a electron transmission probability function, T, can be used to determine the contribution of each DNA base pair. The measured conductance, M, through a section of dsDNA in the transverse direction is calculated by following the equation:

$$Δ σ ( x ) = C + {\sum }_{i}^{j} ( {σ}_{x} * {T}_{x} )$$

where, C is the background baseline shift, σₓ is the conductance of each DNA base pair, Tₓ is the transmission probability based on the location of the DNA base pair

$${T}_{x} = \frac{{ℏ}^{2} {v}^{2}}{{ℏ}^{2} {v}^{2} + {U}^{2}}$$

where, h is the reduced Planck number, v is the applied potential bias, and U is an evaluating number in the range from 0 to 1 for describing the alignment position. U=0 when the DNA base pair is in the middle of the nanoelectrodes where the transmission probability Tₓ=1. After this step, the conductance of each DNA base pair, stored in arrays of σₜ(x), were converted to measurement conductance, stored in arrays of Δσₜ(x).

At step 5, dedicated numbers are used to describe the system conductance change numerically. After this step, the reference map is ready to be used. In this final step of reference map construction, the theoretically established measurement conductance arrays Δσₜ(x) are used. In order to find a match between experimental data and theoretical data, the change of amplitude rather than the absolute value of the amplitude must be used. To accommodate the computer processing requirement, the change of the theoretical data must be described numerically using the following equations:

$${Ψ}_{t} ( x ) = \{ \begin{matrix}4 ; & if Δσ ( x + 1 ) - Δ σ ( x ) > 0 ; \\ 2 ; & if Δσ ( x + 1 ) - Δ σ ( x ) = 0 \\ 0 : & if Δσ ( x + 1 ) - Δ σ ( x ) < 0 ;\end{matrix}$$

where, the array Δσ(x) is the measured conductance based on a group of DNA base pairs appearing in the nanoelectrodes detection range. After this process, in the reference map, the change of the system conductance due to the translocating DNA is expressed numerically without a physical vector. In this way, each time the DNA moved forward one base pair distance, the measurement conductance change of increase, decrease, and flat were represented by the number of 4, 0, and 2, respectively. For a DNA translocating through the gap of sensing nanoelectrodes, a series of numbers is generated to describe the change of measured conductance due to this translocation event. Once this process is repeated on all conductance arrays, the reference maps are prepared and ready to be used. An example reference map is shown in FIG. 14.

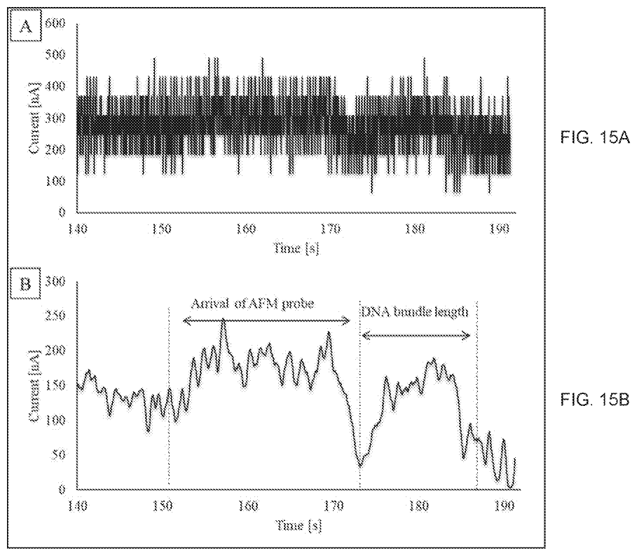

Step 6 is the process of experimental preparation to interpret experimental electrical current change numerically in the same way as the reference map described. That is, experimental data processing follows the same principle by converting data to a series of numbers that represent the change of tunneling current. To do this, a section of experimental data where the DNA is believed to be stretched is selected for analysis. In one embodiment, it may necessary to process the raw data to reduce noise level. For example, noise level may be reduced using a 3rd-order Butterworth LPF with a cutoff frequency of 45 Hz or a Keithley 6485 with a sampling frequency of 1000 Hz. It is contemplated that various equipment or working conditions may be used as known in the art for reducing noise level of the data, and that the particular frequency and other parameters should be modified according to the particular equipment used. In any event, the noise reduction used in this step is only for the purpose of finding the stretched DNA sections. Data before and after processing is shown, for example, in FIG. 15.

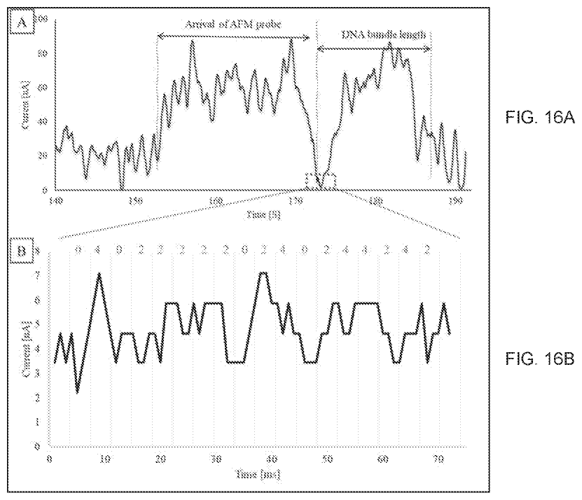

After data processing, the DNA tunneling current is plotted. An example is shown in FIG. 16A. Based on the DNA translocation speed, the data is divided into small sections to obtain the average in each of these sections. For example, if the DNA is translocating at 0.1 μm/s, the experimental data is dissected into small sections with the length of 3 ms for each section. This is based on the fact that the distance between the two neighbor DNA base pairs is 0.3 nm. With a 3 ms interval, theoretically, there will be 1 DNA base pair in each data section. The average is then taken in each section, and the conductance change from one section to another can be numbered by following the same principle. The numbering and its matching results are shown in FIG. 16B as an example. As the example shows, the coded sequence (also referred to in the application as a listing) could be written as [040222220240244242].

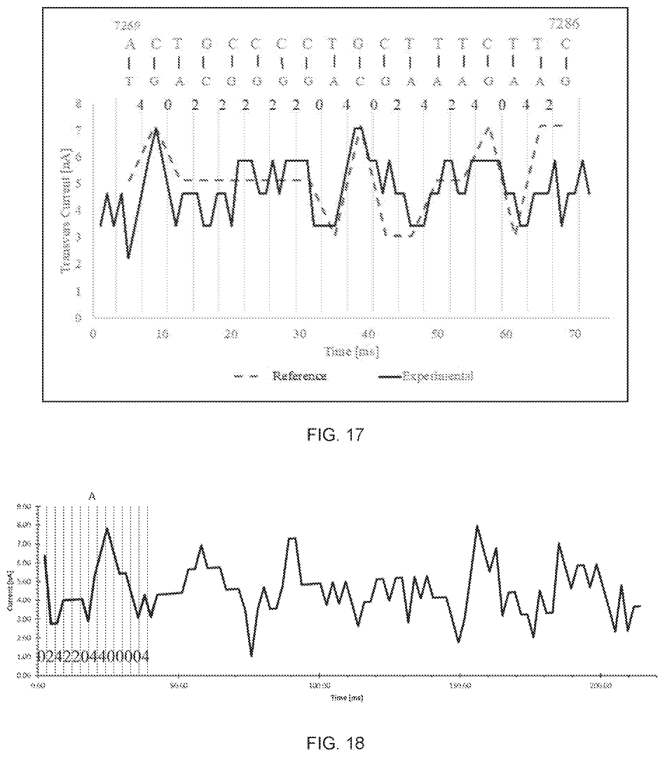

In the seventh step, the processed experimental data is used to find a match on the reference map. The obtained result indicates the position of that matching, which is used to retrieve the sequence information from the standard DNA sequence database. Thus, the developed DNA base calling method identifies the sequenced DNA by conducting a match study between the experimental data and theoretical reference maps (as shown in FIG. 17). In the example above, the coded sequence of [040222220240244242] is used to find a match in the matrices. DNA base calling is then accomplished by finding the corresponding DNA base information by reverse tracing the matrix of measurable conductance (Aa) to the original DNA matrix. After this process, the position of the DNA sequenced in this experiment is located. As shown in FIG. 17, by tracing the reference map, sequence information of [ACTGCCCCTGCTTTCTTC] is located.

In order to carry out the method, it may be seen that the following must be known: (a) the target DNA, (b) the DNA translocation speed, and (c) the width of the nanoelectrodes. Instead of directly ‘reading’ DNA sequence through challenging the current fabrication technology limitation to have a sub-0.3 nm wide sensing nanoelectrodes, the method of the present invention significantly reduces the cost of sequencing for applications where DNA identification is desired. It may be seen that the method of the present invention may be directly used by or embedded in a deep learning algorithm to work with sophisticated mathematical models for further analysis.

Using Experimental Data to Estimate the Accuracy of the DNA Base-Calling Process: As described below, additional experimental data was employed to describe the method of DNA base-calling accuracy determination. A long DNA sequence raw data from a piece of ADNA is partially coded and plotted in FIG. 18. The coding process was conducted based on the signal coding and code matching method described above. Based on the DNA translocation speed, the beginning of this serial data was coded within a time interval of 3 ms. The goal of this first step data coding is to determine the length of the first section, noted as section A, with no mismatching. Then, referring back to the coding matrix described above, the sequencing of section A is found as [CCACGCGGGATGA].

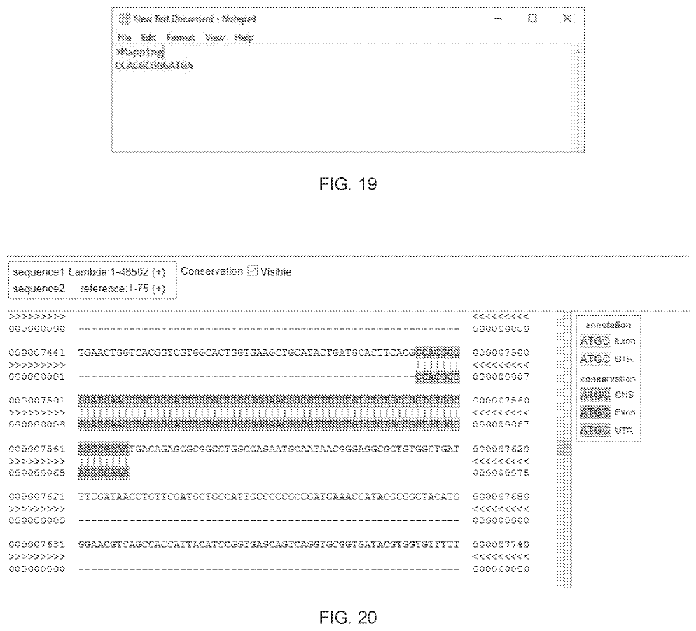

To determine the gene information for the sequenced section of the ADNA starting with the sequencing of [CCACGCGGGATGA], the DNA mapping techniques were carried out using the VISTA tool. The DNA sequence information of the section A was managed into a text file in the format shown in FIG. 19. The name of the sequence was defined by a text string starting with a ‘>’ mark. After the success of the file submission, the mapping result was returned and shown in FIG. 20. The highlighted VISTA mapping result suggests the direction of coding of the rest of the raw data shown in FIG. 18.

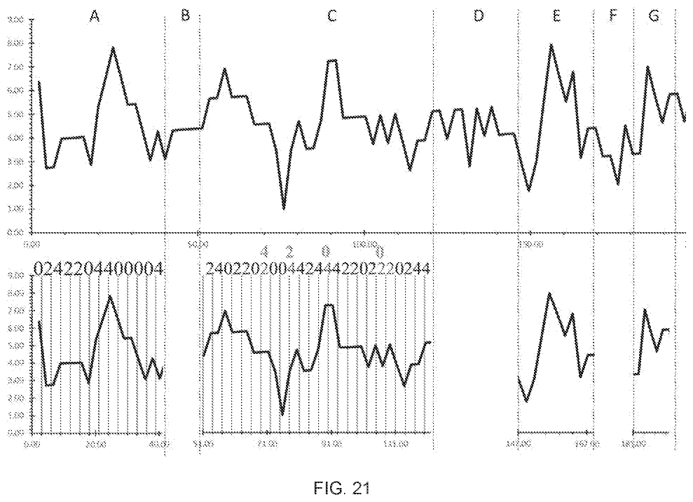

The rest of the data shown in FIG. 18 was coded based on the suggestion of the VISTA mapping result, and the coding result is shown in FIG. 21. The redundant sequence found in sections of B, D, F was determined because it repeated the data found in sections either before or after it. Section C can only be correctly coded if using the time interval of 2.6 ms. Compared to the 3 ms time interval used in section A, the section C is physically less stretched. Some of the numbers of coding result in section C represent errors. For each error, the number on top of the error code represents the corrections suggested by the VISTA mapping results. As shown in FIG. 21 (starting from left to right), a “2” has been corrected to a “4,” a “4” has been corrected to a “2,” a “4” has been corrected to a “0,” and a “2” has been corrected to a “0.” As a result, the data section A contains the DNA sequence of [CCACGCGGGATGA], and the section C contains the sequence of [ACCTGTGGCATTTGTGCTGCCGGGAAC] after the correction.

In order to successfully code sections E and G, the time interval of 1 ms has to be employed. For this particular group of experimental data, the 1 ms time interval reaches to the limit of the DAQ system used for data collection which only has a 1000 Hz maximum sampling frequency and causes the raw data to be unsuitable for further analysis. Therefore, the seqeunce data in sections E and G will not be counted for determing the base-calling accuracy.

In summary, in this particular group of DNA sequencing data, a total length of 40 base pair DNA was successfully processed using the disclosed base-calling method with 4 errors, which suggests a 90.47% local accuracy using the equation:

$$ɛ = 1 - \frac{δ}{N}$$

where, ε is simply the accuracy, δ is the count of errors, and N is the total number of DNA sequence embedded in the raw data. The DNA sections E and G were not counted as a successful processing result due to the limited sampling frequency. In section E, there were 21 base pairs during the time of 21 ms. Similarly, in section G, there were 14 base pairs DNA packed in a time duration of 13 ms.

In a macro scale, the success rate of the DNA sequencing result was low by giving a total of 36 base pairs correctly read out from a 75 bp DNA segment. It roughly gives the global accuracy of 48%. Though 48% is not a significant number, it still shows the potential when considering the 65% raw accuracy of Oxford Nanopore MinION that has been developed for a decade.

The improvement for using this disclosed DNA base-calling accuracy can be achieved from two major perspectives. The most obvious way for achieving higher base-calling accuracy is to use an advanced DAQ system with a higher sampling frequency. In this particular example, the major fall back of the global accuracy is due to the limit of the DAQ system. The other improvement can be realized through the dimension reduction of the sensing element to improve the signal to noise ratio. The data used in this study as an example was measured using a 100 nm wide nanoelectrodes. The changing of the conductance caused by the translocating DNA was described above using the equation of:

$$Δ σ ( x ) = C + {\sum }_{i}^{j} ( {σ}_{x} * {T}_{x} )$$

With a 1 nm width reduction of the nanoelectrodes, the Δσ is reduced by ˜1% in average. The change of the signal to noise ratio can be described using the following equation:

$$Δ α ≅ \frac{Δ σ ( x )}{{Δσ}^{′} ( x )} - 1$$

where the Δα is the improvement of the signal to noise ratio and Δσ′(x) is the overall conductance measured by nanoelectrodes with a reduced width. Based on the equations, reducing the width of nanoelectrodes from the current 100 nm to 50 nm will double the signal to noise ratio. The connect between the improved signal to noise ratio and the overall DNA base-calling accuracy enhancement is still under investigation.

The present invention has been described with reference to certain preferred and alternative embodiments that are intended to be exemplary only and not limiting to the full scope of the present invention.

We claim:

1. A method of DNA-base calling, comprising the steps of:
(a) building a reference map comprising reference characters corresponding to a change in conductance measured as a known sequence of double-stranded DNA is translocated through nanoelectrodes of a DNA sequencer;
(b) determining conductance of an unknown sequence of double-stranded DNA measured as said unknown sequence of double-stranded DNA is translated through said nanoelectrodes of said DNA sequencer
(c) determining changes in said conductance between adjacent sections of said unknown sequence of double-stranded DNA;
(d) assigning reference characters corresponding to said changes in conductance to create a listing;
(e) matching said listing to said reference map; and
(f) determining a sequence of said unknown sequence of double-stranded DNA based on said matching of said listing to said reference map.

2. The method of claim 1, wherein said reference characters of said reference map and said reference characters of said listing are numerals.

3. The method of claim 1, wherein said step of building a reference map comprises the steps of:
(a) converting a known sequence of single-stranded DNA to said double-stranded DNA comprising a plurality of base pairs;
(b) determining orientations of said plurality of base pairs relative to a first base pair of said plurality of base pairs;
(c) calculating an equivalent conductance of each of said plurality of base pairs of said double-stranded DNA based on said orientations of said plurality of base pairs;
(d) calculating system conductances of adjacent sections of said double-stranded DNA; and
(e) assigning said reference characters of said reference map corresponding to changes in said system conductances in adjacent sections of said double-stranded DNA.

4. The method of claim 2, further comprising the step of building a matrix comprising said known sequence of said double-stranded DNA and said orientation of said plurality of base pairs.

5. The method of claim 2, wherein said step of calculating an equivalent conductance of each of said plurality of base pairs of said double-stranded DNA comprises the step of selecting a formula based on said orientations of said plurality of base pairs.

6. The method of claim 2, wherein said system conductances are equal to a sum of said equivalent conductance of each of said plurality of base pairs within a detection range of said nanoelectrodes.

7. The method of claim 6, wherein said detection range of said nanoelectrodes is determined by a width of said nanoelectrodes.

8. The method of claim 1, wherein prior to step (b) noise reduction is performed on said unknown sequence of double-stranded DNA.

9. The method of claim 1, wherein prior to step (c) said conductance of said unknown sequence of double-stranded DNA is plotted.
